(19) United States
(12) Patent Application Publication
(10) Pub. No.: US 2026/0054042 A1
(43) Pub. Date: Feb. 26, 2026

(54) APPARATUS AND METHOD OF APPLYING MEDICATION

(71) Applicant: Hope Innovations, LLC, Audubon, IA (US)

(72) Inventors: Hope J. Jensen, Audubon, IA (US); Elizabeth Thornton, Scarborough, ME (US); Andrew Powell, Somerville, MA (US)

(21) Appl. No.: 19/307,294

(22) Filed: Aug. 22, 2025

Related U.S. Application Data
(60) Provisional application No. 63/685,847, filed on Aug. 22, 2024.
(60) Provisional application No. 63/759,416, filed on Feb. 17, 2025.

(51) Int. Cl. A61M 31/00

(52) U.S. Cl. CPC A61M 31/007; A61M 2202/0007; A61M 2205/0216; A61M 2210/1475

(57) ABSTRACT

Various applicators are disclosed. The applicators may be used to deposit medication inside a vagina. In one embodiment, the applicator includes a tube and a bulb. The tube has a stiff cylindrical body having one end connected to the bulb and a flexible membrane arranged at a another end of the tube. The applicator is generally airtight and the orientation of the flexible membrane is controlled by squeezing the bulb. When the bulb is squeezed the flexible membrane inverts to outside the body when the bulb is released the flexible membrane and returns to inside the body.

### CROSS REFERENCE TO RELATED APPLICATIONS

This application claims the benefit of U.S. Provisional Patent Application No. 63/685,847 which was filed with the United States Patent and Trademark Office on Aug. 22, 2024 and U.S. Provisional Patent Application No. 63/759,416 which was filed with the United States Patent and Trademark Office on Feb. 17, 2025, the entire contents of each of which are herein incorporated by reference.

### BACKGROUND

### 1. Field

Example embodiments relate to an intravaginal medication applicator and method for intravaginal application of medication.

### 2. Description of the Related Art

Intravaginal medication applicators are used to apply medication inside a vagina. Conventional intravaginal medication applicators typically include a syringe type applicator having a cylinder and a plunger to push medication out of the cylinder. In these applicators, medication is pre-loaded into the applicator, the applicator is inserted into the vagina, and the medication is deployed from the applicator. Having the correct intravaginal application of medicine can be important for several reasons. For example, the ability to deploy medication intravaginally can allow diseases and conditions of the vagina to be treated locally, providing a proper concentration of medication at the site of the disease or condition.

### SUMMARY

The inventors have noted several problems with conventional intravaginal medication applicators. First, the medication applicators do not accommodate all the different forms of medication. The variance in type and form of different intravaginal medications require different types of applicators to be used with different types and forms of medication. Second, the medication applicators can be difficult to clean after each use. Third, the medication applicators may not be used entirely with one hand. Fourth, many women report a conventional applicator that comes with estrogen causes a ball, chunk, lump, or clump of cream that can sometimes release out of the vagina in one piece and is messy. As a consequence, the inventors set out to solve these problems. The result is a modified intravaginal medication applicator which virtually eliminates the aforementioned problems. In some embodiments of the applicator, a plunger element is not needed to intravaginally dispense medication.

In example embodiments, an intravaginal medication applicator may have a tube with a first end and a second end. In example embodiments, the intravaginal medication applicator may have a flexible member encasing the tube having the first end and the second end. In example embodiments, the first end of the flexible member may have a first position and a second position. The ends of the flexible member may cover the ends of the tube creating an air chamber inside the applicator. The first position of the first end of the flexible member may be inside the first end of the tube which may create a medication chamber. When the first end of the flexible member is moved into the first position, an air pocket may be created at the second end of the flexible member. A medication may be inserted into the medication chamber. When the air pocket is pressed by a user and the second end of the flexible member is inverted into the second end of the tube, air inside the air chamber may displace the first end of the flexible member from the first position to the second position.

In example embodiments, an intravaginal medication applicator may have an enclosed tube. The enclosed tube may have a middle portion, a first end, and a second end. The middle portion of the tube may be rigid, and the first end and the second end of the tube may be flexible. An air chamber may be located inside of the enclosed tube. The first end of the tube may have a first position and a second position, the first position being inverted inside of the middle portion of the tube and the second position being outside of the tube. When the first end is inverted in the first position, the second end may be outside of the tube. A medication may be loaded into the applicator when the first end of the tube is in the first position. When a user applies pressure to the second end, the air chamber may facilitate inflection of the first end from the first position to the second position, allowing application of the medication.

In example embodiments, a method of intravaginal medication application is provided. The method may include positioning a first end of an enclosed tube to an inverted position inside of the tube to create a medication chamber. The method may also include a step of applying and/or placing a medication into the medication chamber. The tube may be inserted inside a vagina to a desired intravaginal depth. By pressing a second end of the tube to an inverted position, the first end may be inflected out of the inverted position and into a deployed position. When the first end is in the deployed position the medication can be/or is transferred to a desired intravaginal area. The tube may be removed from the vagina.

Disclosed also is an applicator comprising a tube and a bulb. In one nonlimiting example embodiment the tube has a stiff body and a flexible membrane, where the body includes a first end and a second end, where the first end of the body is open and the flexible membrane is arranged at the second end of the body. In this nonlimiting example embodiment, the flexible membrane may be in a first state where the flexible membrane resides in the body and a second state where the flexible membrane resides outside of the body. The bulb may be attached the second end of the body, wherein when the bulb is squeezed air in the applicator is moved against the flexible membrane to cause the flexible membrane to be in the second state and when the bulb is released air moves back into the bulb causing the flexible membrane to invert back to the first state.

Disclosed also is a method of applying medication. In this example embodiment a user obtains an applicator having a tube and a bulb, wherein the tube has a body having a first end connected to the bulb and a second end with a flexible membrane. In this nonlimiting example embodiment the flexible membrane may be in a first state where the flexible membrane resides in the body and a second state where the flexible membrane resides outside of the body. In this method medication placed on the flexible membrane while the flexible membrane resides in the body. Thereafter the applicator is inserted into the vagina and the flexible member is inverted by squeezing the bulb. Inversion of the flexible member places the medication inside the vagina.

### BRIEF DESCRIPTION OF THE DRAWINGS

Example embodiments are described in detail below with reference to the attached drawing figures, wherein:

FIG. 1 is a first perspective view of an intravaginal medication applicator in accordance with example embodiments;

FIG. 2 is a first cross-section view of an intravaginal medication applicator in accordance with example embodiments;

FIG. 3 a second cross-section view of an intravaginal medication applicator in accordance with example embodiments;

FIG. 4 is a first cross-section view of the tube in accordance with example embodiments;

FIG. 5 is a first isometric view of the tube in accordance with example embodiments;

FIG. 6 is a third cross-section view of an intravaginal medication applicator illustrating a medication loaded into the applicator in accordance with example embodiments;

FIG. 7 illustrates a view of an intravaginal medication applicator inserted into a vagina in accordance with example embodiments;

FIG. 8 illustrates a view of an intravaginal medication applicator being deployed in a vagina in accordance with example embodiments;

FIG. 9 is a second cross-section view of the tube in accordance with example embodiments;

FIG. 10 is a third cross-section view of the tube in accordance with example embodiments;

FIG. 11 is a fourth cross-section view of the tube in accordance with example embodiments;

FIG. 12 is a fifth cross-section view of the tube in accordance with example embodiments;

FIG. 13 is a second perspective view of an intravaginal medication applicator in accordance with example embodiments;

FIG. 14 is a fourth cross-section view of an intravaginal medication applicator in accordance with example embodiments;

FIG. 15 a fifth cross-section view of an intravaginal medication applicator in accordance with example embodiments;

FIG. 16 is a sixth cross-section view of the tube in accordance with example embodiments;

FIG. 17 is a second isometric view of the tube in accordance with example embodiments;

FIG. 18 is a sixth cross-section view of an intravaginal medication applicator illustrating a medication loaded into the applicator in accordance with example embodiments;

FIG. 19 illustrates a view of an intravaginal medication applicator inserted into a vagina in accordance with example embodiments;

FIG. 20 illustrates a view of an intravaginal medication applicator being deployed in a vagina in accordance with example embodiments;

FIG. 21 illustrates a seventh cross-section view of the tube in accordance with example embodiments;

FIG. 22 is a seventh cross-section view of an intravaginal medication applicator in accordance with example embodiments;

FIG. 23 is a third isometric view of the tube in accordance with example embodiments;

FIG. 24 is a fourth isometric view of the tube in accordance with example embodiments;

FIG. 25 is a fifth isometric view of the tube in accordance with example embodiments;

FIG. 26 is an isometric view of an intravaginal medication applicator in accordance with example embodiments;

FIG. 27 illustrates a finger deploying an intravaginal medication applicator in accordance with example embodiments;

FIG. 28 illustrates an intravaginal medication applicator in a deployed state in accordance with example embodiments;

FIG. 29 illustrates a perspective view of another intravaginal medication applicator in accordance with example embodiments;

FIG. 30 illustrates another perspective view of the intravaginal medication applicator in accordance with example embodiments;

FIG. 31 illustrates a first side view of the intravaginal medication applicator in accordance with example embodiments;

FIG. 32 illustrates a second side view of the intravaginal medication applicator in accordance with example embodiments;

FIG. 33 illustrates a first cross-section side view of the intravaginal medication applicator in accordance with example embodiments;

FIG. 34 illustrates a second cross-section side view of the intravaginal medication applicator in accordance with example embodiments;

FIG. 35 illustrates an exploded view of the exemplary intravaginal medication applicator in accordance with example embodiments;

FIG. 36 illustrates an exploded view of the exemplary intravaginal medication applicator in accordance with example embodiments;

FIG. 37 is a close up view of the exemplary intravaginal medication applicator in accordance with example embodiments;

FIG. 38 is another close up view of the exemplary intravaginal medication applicator in accordance with example embodiments;

FIG. 39 is an example of an operation associated with the exemplary intravaginal medication applicator in accordance with example embodiments;

FIG. 40 is an example of another operation associated with the exemplary intravaginal medication applicator in accordance with example embodiments;

FIG. 41 is an example of another operation associated with the exemplary intravaginal medication applicator in accordance with example embodiments; and

FIG. 42 is an example of another operation associated with the exemplary intravaginal medication applicator in accordance with example embodiments.

### DETAILED DESCRIPTION

Example embodiments will now be described more fully with reference to the accompanying drawings, in which example embodiments of the invention are shown. The invention may, however, be embodied in different forms and should not be construed as limited to the embodiments set forth herein. Rather, these embodiments are provided so that this disclosure will be thorough and complete, and will fully convey the scope of the invention to those skilled in the art. In the drawings, the sizes of components may be exaggerated for clarity.

It will be understood that when an element or layer is referred to as being “on,” “connected to,” or “coupled to” another element or layer, it can be directly on, connected to, or coupled to the other element or layer or intervening elements or layers that may be present. In contrast, when an element is referred to as being “directly on,” “directly connected to,” or “directly coupled to” another element or layer, there are no intervening elements or layers present. As used herein, the term “and/or” includes any and all combinations of one or more of the associated listed items.

It will be understood that, although the terms first, second, etc. may be used herein to describe various elements, components, regions, layers, and/or sections, these elements, components, regions, layers, and/or sections should not be limited by these terms. These terms are only used to distinguish one element, component, region, layer, and/or section from another elements, component, region, layer, and/or section. Thus, a first element component region, layer or section discussed below could be termed a second element, component, region, layer, or section without departing from the teachings of example embodiments.

Spatially relative terms, such as “beneath,” “below,” “lower,” “above,” “upper,” and the like, may be used herein for ease of description to describe one element or feature's relationship to another element(s) or feature(s) as illustrated in the figures. It will be understood that the spatially relative terms are intended to encompass different orientations of the structure in use or operation in addition to the orientation depicted in the figures. For example, if the structure in the figures is turned over, elements described as “below” or “beneath” other elements or features would then be oriented “above” the other elements or features. Thus, the exemplary term “below” can encompass both an orientation of above and below. The structure may be otherwise oriented (rotated 90 degrees or at other orientations) and the spatially relative descriptors used herein interpreted accordingly.

Embodiments described herein will refer to plan views and/or cross-sectional views by way of ideal schematic views. Accordingly, the views may be modified depending on manufacturing technologies and/or tolerances. Therefore, example embodiments are not limited to those shown in the views, but include modifications in configurations formed on the basis of manufacturing process. Therefore, regions exemplified in the figures have schematic properties and shapes of regions shown in the figures exemplify specific shapes or regions of elements, and do not limit example embodiments.

The subject matter of example embodiments, as disclosed herein, is described with specificity to meet statutory requirements. However, the description itself is not intended to limit the scope of this patent. Rather, the inventors have contemplated that the claimed subject matter might also be embodied in other ways, to include different features or combinations of features similar to the ones described in this document, in conjunction with other technologies. Generally, example embodiments relate to an intravaginal medication applicator usable for applying medication inside a vagina.

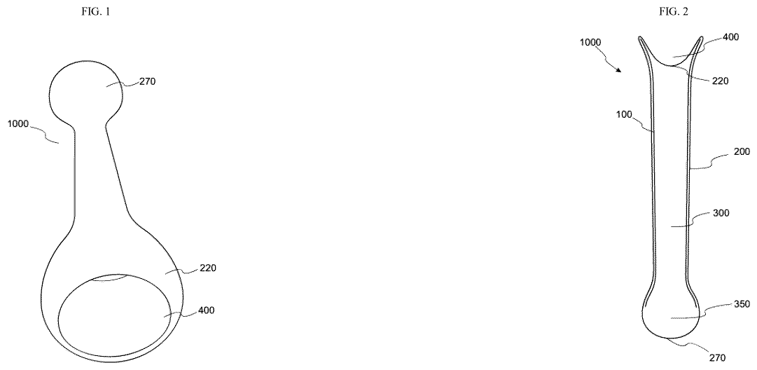

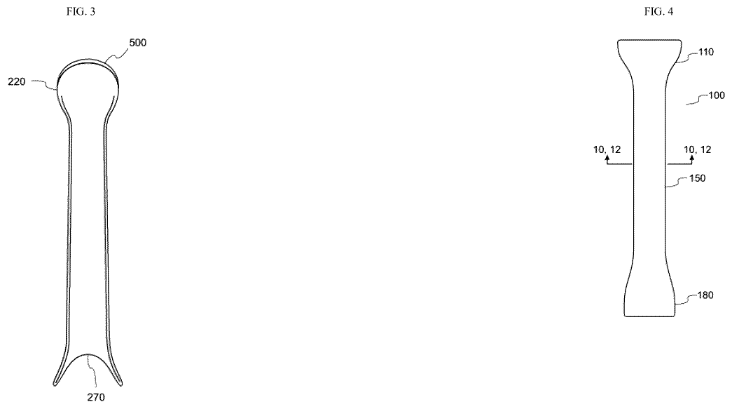

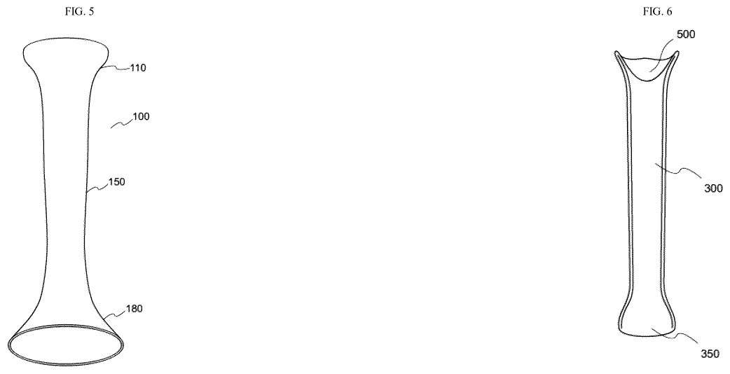

FIG. 1 is a first perspective view of an intravaginal medication applicator 1000 usable for applying medication 500 to a vagina 600. Referring to FIGS. 1-8, the intravaginal medication applicator 1000 according to a nonlimiting example embodiment is comprised of a tube 100 and a flexible member 200. FIG. 2 is a first cross-section view of the intravaginal medication applicator 1000. FIG. 3 a second cross-section view of the intravaginal medication applicator 1000. FIG. 4 is a first cross-section view of the tube 100. FIG. 5 is a first isometric view of the tube 100. FIGS. 6-8 illustrate the process of using the intravaginal medication applicator 1000 to apply medication 500 to a vagina 600.

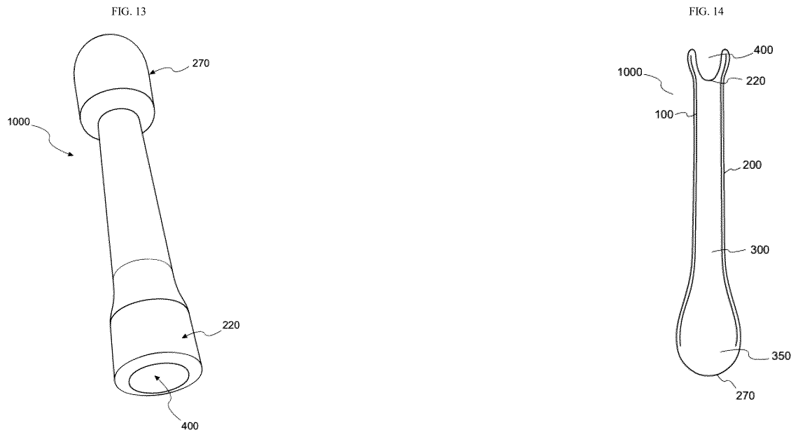

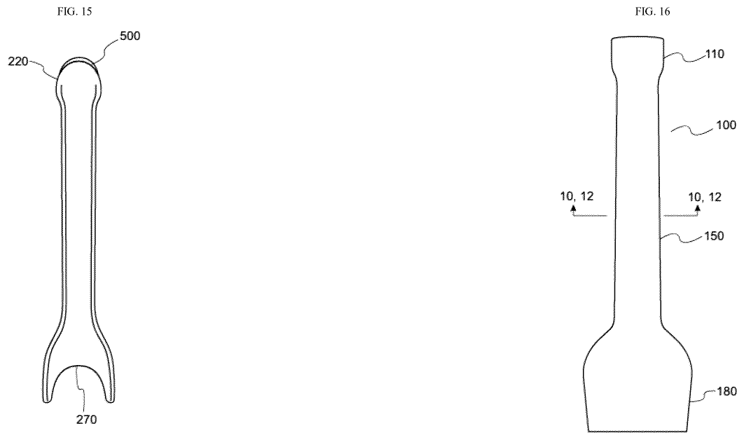

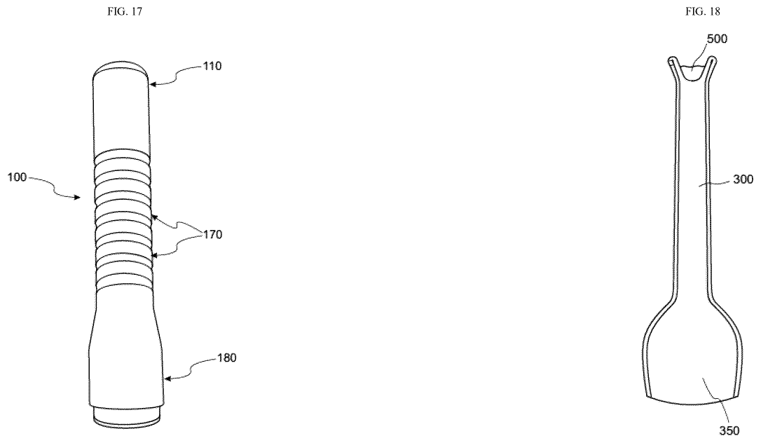

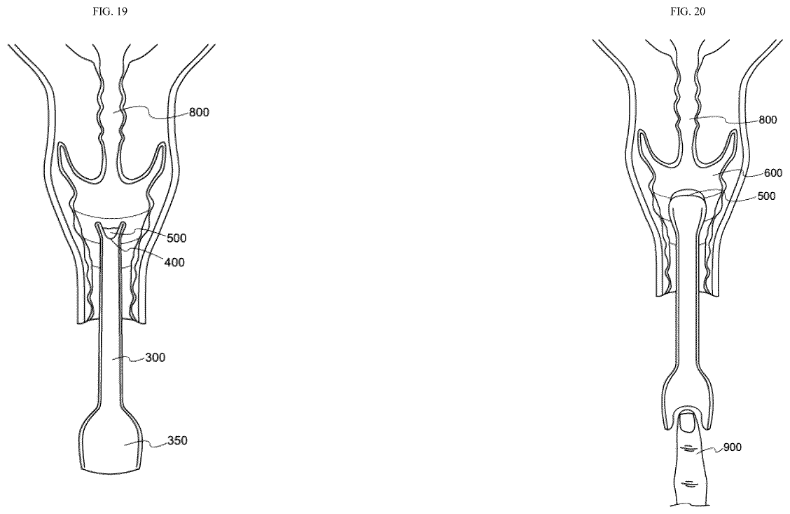

FIG. 13 is a second perspective view of an intravaginal medication applicator 1000 usable for applying medication 500 to a vagina 600. Referring to FIGS. 13-20, the intravaginal medication applicator 1000 according to a nonlimiting example embodiment is comprised of a tube 100 and a flexible member 200. FIG. 14 is a fourth cross-section view of the intravaginal medication applicator 1000. FIG. 15 a fifth cross-section view of the intravaginal medication applicator 1000. FIG. 16 is a sixth cross-section view of the tube 100. FIG. 17 is a second isometric view of the tube 100. FIGS. 18-20 illustrate the process of using the intravaginal medication applicator 1000 to apply medication 500 to a vagina 600.

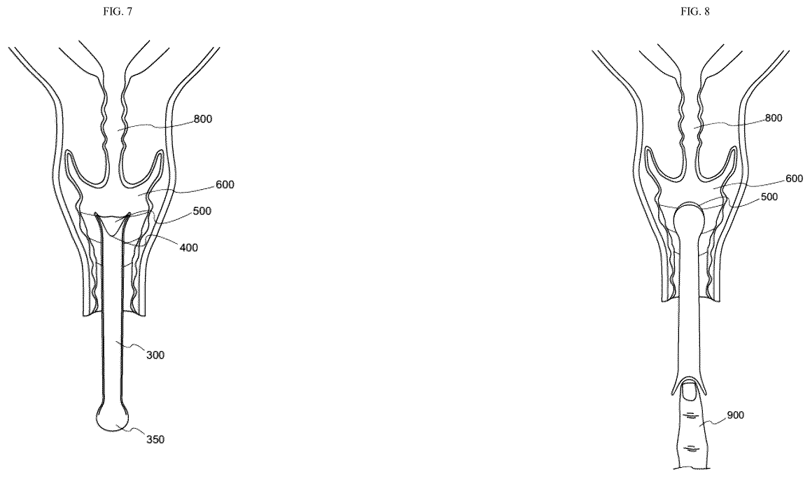

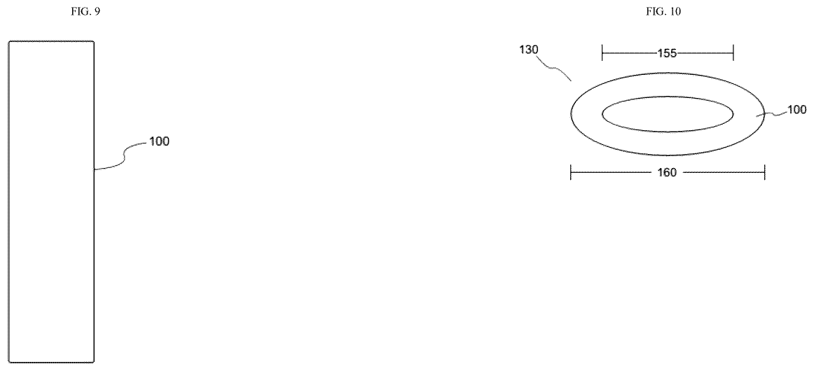

Referring to FIG. 4, the tube 100 may include a first end 110, a middle portion 150, and a second end 180. The tube 100 may be configured to provide structural integrity to the intravaginal medication applicator 1000 and facilitate insertion of the intravaginal medication applicator 1000 into a vagina 600 (see FIG. 7). The tube 100 may be hollow to permit air 320 to move freely through the middle portion 150 between the first end 110 and the second end 180. The cross section 130 of the tube 100 may have an inner diameter 155 and an outer diameter 160. The cross section 130 of the tube 100 may be round to facilitate insertion into a vagina 600. For example, in one embodiment, the cross section 130 of the tube 100 is the shape of a circle and/or annulus (see FIG. 12). In another embodiment, the cross section 130 of the tube 100 is the shape of an ellipse (see FIG. 10). The cross section 130 of the tube 100 can be optimized to function with different types, shapes, and sizes of medication 500. For example, potential types of medication 500 could include a pill, a tablet, a solution, a cream, a gel, a ring, or a suppository. In yet another embodiment, the middle portion 150 of the tube 100 may have ridges 170 to aid a user 950 in gripping the medication applicator 1000 (see FIG. 17, 22-23).

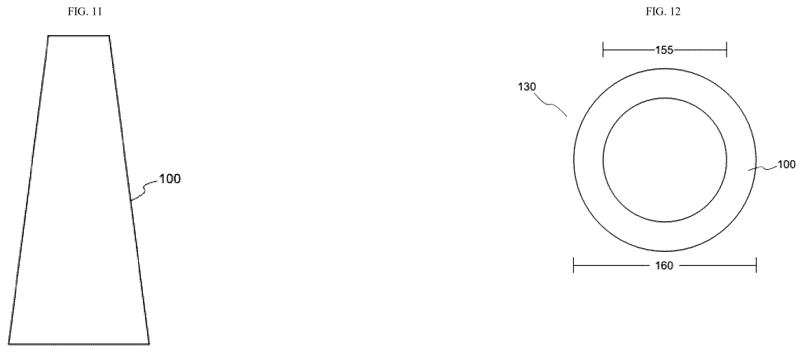

In example embodiments, the first end 110 of the tube 100 of the intravaginal medication applicator 1000 may be configured to provide structural support to a first end 220 of the flexible member 200 to facilitate application of a medication 500 to a vagina 600. For example, in one embodiment, the first end 110 of the tube 100 may be larger in diameter than the middle portion 150 of the tube 100 (see FIG. 4). This, however, is not meant to limit the invention since, in another embodiment, the first end 110 of the tube 100 may be equal in diameter to the middle portion 150 of the tube 100 (see FIG. 9). In yet another embodiment, the first end 110 of the tube 100 may be smaller in diameter than the middle portion 150 of the tube 100 (see FIG. 11).

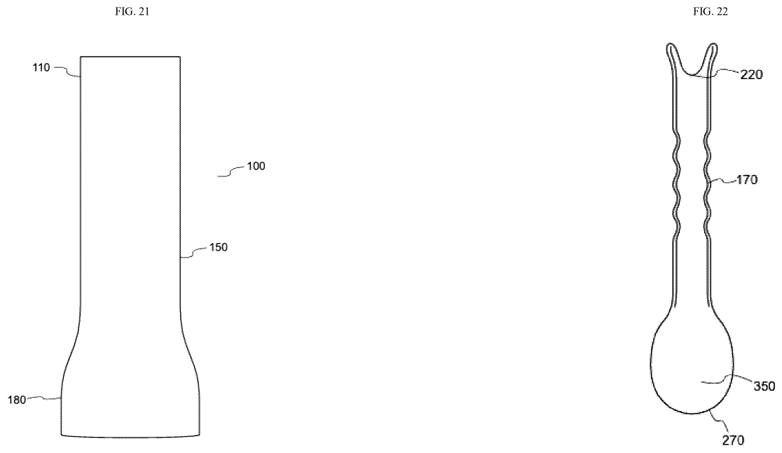

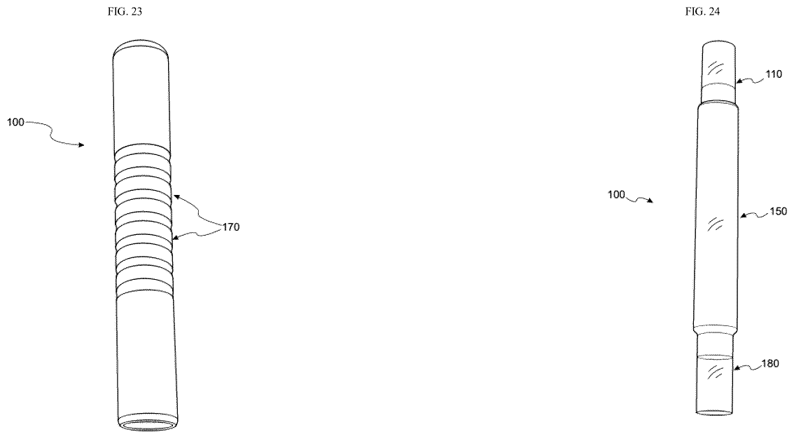

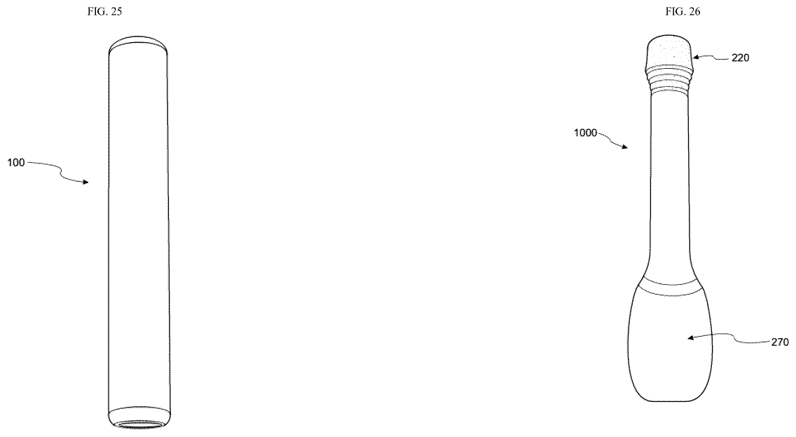

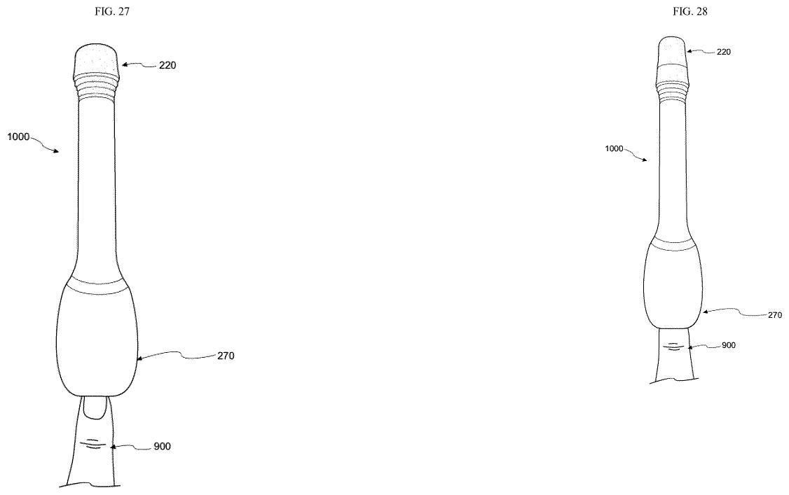

In example embodiments, the second end 180 of the tube 100 of the intravaginal medication applicator 1000 may be configured to provide structural support to a second end 270 of the flexible member 200 to facilitate movement of air 320 through an air chamber 300. For example, in one embodiment, the second end 180 of the tube 100 may be larger in diameter than the middle portion 150 of the tube 100 (see FIG. 11). In another embodiment, the second end 180 of the tube 100 may be equal in diameter to the middle portion 150 of the tube 100 (see FIG. 9). In another embodiment, the second end 180 of the tube 100 may be equal in diameter to the first end 110 of the tube 100, which each have a diameter greater than the middle portion 150 of the tube 100 (see FIG. 4). In another embodiment, the second end 180 of the tube 100 may be greater in diameter than the first end 110 of the tube 100, which each have a diameter greater than the middle portion 150 of the tube 100 (see FIG. 16). In another embodiment, the second end 180 of the tube 100 may be greater in diameter than the first end 110 of the tube 100, which the first end 110 of the tube 100 is equal in diameter to the middle portion 150 of the tube 100 (see FIG. 21). The second end 180 of the tube 100 may be greater in diameter than the rest of the tube 100 to better accommodate a user 950 in deploying the intravaginal medication applicator 1000 with their finger 900.

Referring to FIG. 2, the flexible member 200 may be connected to the tube 100 to facilitate application of a medication 500 in a vagina 600. For example, the flexible member 200 may be frictionally connected or connected using an adhesive to the tube 100. In at least one embodiment, the flexible member 200 extends past the first end 110 and the second end 180 of the tube 100. In another embodiment, the flexible member 200 contains multiple pieces that cover the first end 110 and the second end 180 of the tube 110. The flexible member 200 may be made of a material that permits manipulation and flexibility of the shape of the flexible member 200. For example, in one embodiment, the flexible member 200 is made of a medical-grade silicon.

In example embodiments, the first end 220 of the flexible member 200 encloses the first end 110 of the tube 100. The first end 220 of the flexible member 200 may be either concave or convex in position with respect to the first end 110 of the tube 100. When the first end 220 of the flexible member 200 is in a concave position, the first end 220 of the flexible member 200 is positioned inside of the first end 110 of the tube 100. When the first end 220 of the flexible member 200 is in a convex position, the first end 220 of the flexible member 200 is positioned outside of the first end 110 of the tube 100. When the first end 220 of the flexible member 200 is in the concave position, a medication chamber 400 is created where a medication 500 can be loaded. When the medication 500 is loaded into the medication chamber 400, the intravaginal medication applicator 1000 may be inserted into the vagina 600. Once inserted inside a vagina 600, the medication chamber 400 created by the first end 220 of the flexible member 200 can be inverted into the convex position by movement of air 320 through the air chamber 300 to facilitate application of the medication 500 to the vagina 600.

In example embodiments, the second end 270 of the flexible member 200 may enclose the second end 180 of the tube 100. The second end 270 of the flexible member 200 may be either concave or convex in position with respect to the second end 180 of the tube 100. When the second end 270 of the flexible member 200 is in a concave position, the second end 270 of the flexible member 200 is positioned inside of the second end 180 of the tube 100. When the second end 270 of the flexible member 200 is in a convex position, the second end 270 of the flexible member 200 is positioned outside of the second end 180 of the tube 100. When the second end 270 of the flexible member 200 is in a convex position, the second end 270 of the flexible member 200 includes an air pocket 350 inside of the second end 270 of the flexible member 200. In another embodiment the second end 270 may be a bulb which may be squeezed to facilitate movement of air 320 through the air chamber 200.

In example embodiments, the second end 270 of the flexible member 200 may enclose the second end 180 of the tube 100, but may not be structurally supported by the second end 180 of the tube 100 (see FIG. 22). The second end 270 of the flexible member 200 may be configured to be bulb shaped. When a user 950 squeezes or presses into the second end 270 of the flexible member 200, air 320 may move through the air chamber 300 and may cause the medication chamber 400 to be deployed into the convex position.

In example embodiments, the movement of air 320 through the air chamber 300 causes the first end 220 and the second end 270 of the flexible member 200 to invert between concave and convex positions. The amount of air 320 inside the intravaginal medication applicator 1000 may allow only one of the first end 220 or the second end 270 of the flexible member 200 to be inverted in position at any one time. When the first end 220 of the flexible member 200 is in a convex position outside of the first end 110 of the tube 100, the second end 270 of the flexible member 200 may be in a concave position inside the second end 180 of the tube 100. Accordingly, when the first end 220 of the flexible member 200 is in a concave position inside of the first end 110 of the tube 100, the second end 270 of the flexible member 200 may be in a convex position outside the second end 180 of the tube 100.

In example embodiments, a finger 900 may be used to press on the second end 270 of the flexible member 200 when it is in a convex position, inverting it to a concave position, to move air 320 from the air pocket 350 through the air chamber 300 to invert the first end 220 of the flexible member 200. Air 320 can be moved freely back and forth through the air chamber 350, creating air pockets 350 at either the first end 220 or the second end 270 of the flexible member 200.

In example embodiments, the method of applying medication 500 to a vagina 600 using the intravaginal medication applicator 1000 is demonstrated. First, a medication 500 is loaded into the medication chamber 400 created by the first end 220 of the flexible member 200 being inverted inside the first end 110 of the tube 100 (see FIG. 6, 18). Second, the intravaginal medication applicator 1000 is inserted into the vagina 600 to a desired depth (see FIG. 7, 19). Third, a finger 900 is used to invert the second end 270 of the flexible member 200 inside the second end 180 of the tube 100 (see FIG. 8, 20). This may cause the air 320 in the air pocket 350 located inside the second end 270 of the flexible member 200 to move through the air chamber 300 and invert the first end 220 of the flexible member 200 to a convex position outside of the first end 110 of the tube 100. When the first end 220 of the flexible member 200 is in the convex position, the medication 500 may be applied to or deposited in the vagina 600. In at least one embodiment, the method of applying medication 500 to a vagina 600 using the intravaginal medication application 1000 can be done with one hand. In another embodiment, the method of applying medication 500 to a vagina 600 using the intravaginal medication application 1000 can be done with more than one hand. For example, in one embodiment, the method of using the intravaginal medication applicator 1000 may be done with two hands.

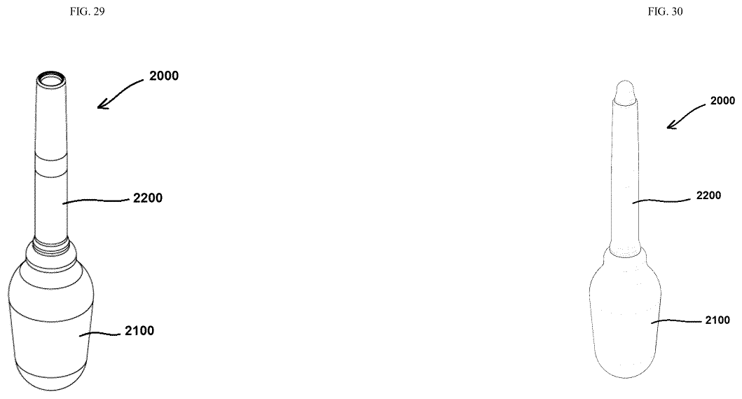

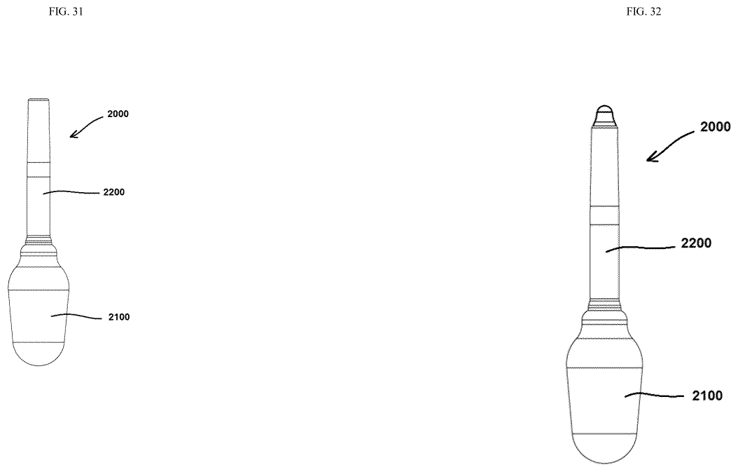

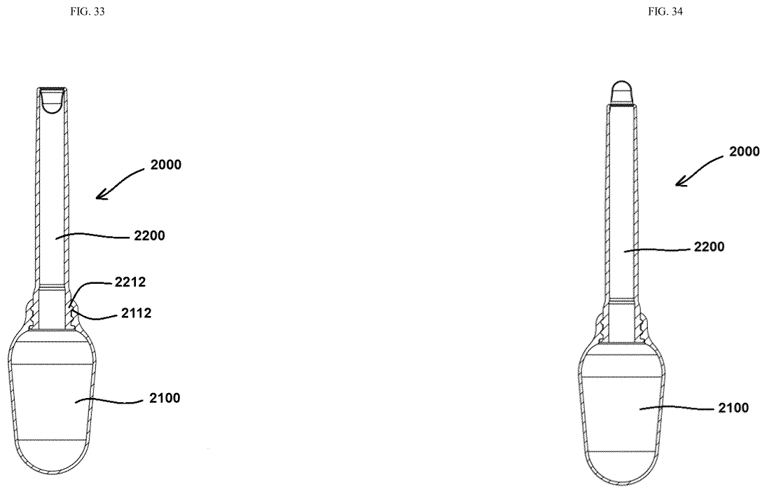

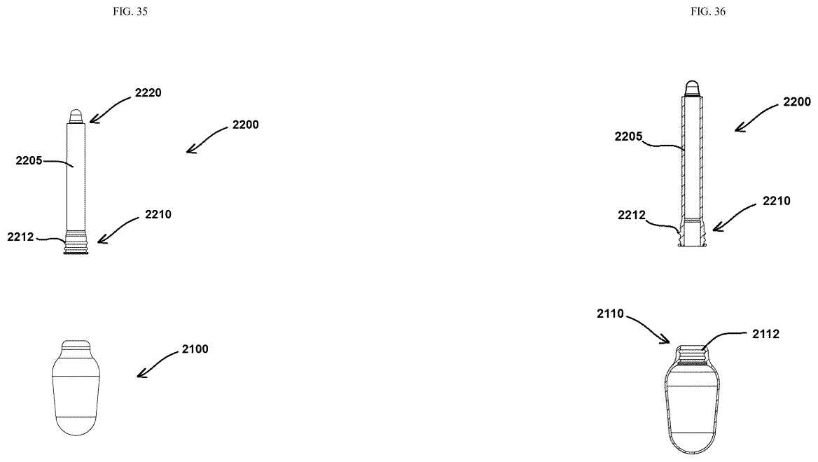

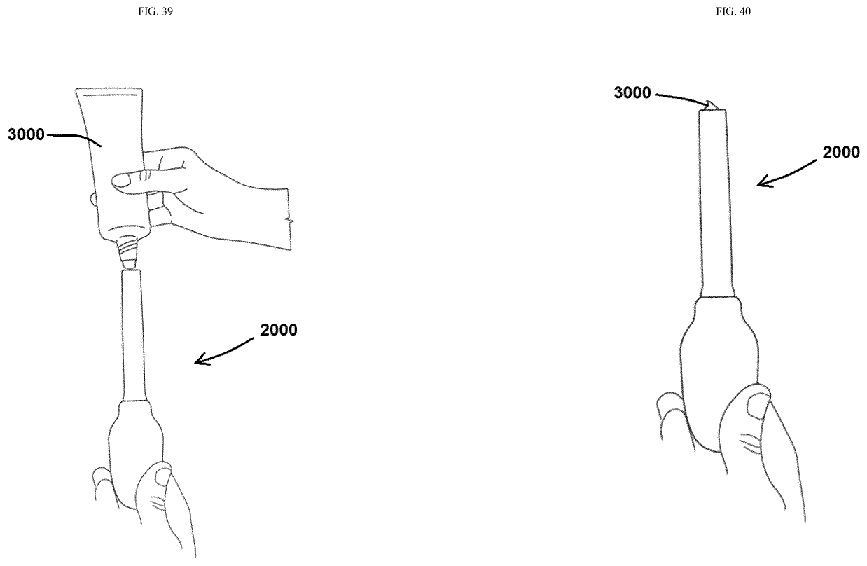

FIGS. 29-42 illustrate yet another example of an applicator (which may be an intravaginal medication applicator) 2000 in accordance with a nonlimiting example of the invention. In particular, FIG. 29 illustrates a perspective view of the applicator 2000 in a first state and FIG. 30 illustrates a perspective view of the applicator 2000 in a second state. FIGS. 31 and 32 illustrate side views of the applicator 2000 in the first state and the second state, respectively. FIGS. 33 and 34 illustrate cross-section side views of the applicator 2000 in the first state and the second state, respectively. FIG. 35 illustrates an exploded view of the exemplary applicator 2000 and FIG. 36 illustrates an exploded view of the exemplary applicator 2000 in cross-section. FIGS. 39-42 illustrate an exemplary use of the applicator 2000 in accordance with a nonlimiting example of the invention.

Referring to FIGS. 29-36 the exemplary applicator 2000 may be comprised of a bulb 2100 attached to a tube 2200. In example embodiments, the bulb 2100 and the tube 2200 may be made from a suitable material, for example, an elastomeric polymer such as silicone rubber, thermoplastic elastomeric polymer for various durometers. In one nonlimiting example embodiment, the bulb 2100 and the tube 2200 may be made from a platinum cured liquid silicone rubber (LSR) which may offer ease of sanitizing the applicator 2000 in boiling water. It is understood the aforementioned materials are for exemplary purposes only and are not meant to limit the invention.

In example embodiments, the bulb 2100 and the tube 2200 may form a substantially air tight structure. In practice, the tube 2200 may include a somewhat stiff body 2205 to allow at least a partial insertion of the applicator 2000 into a vagina, however, the bulb 2100 may be deformable allowing a user to deform the bulb 2100 by grasping the bulb 2100 and squeezing it. In example embodiments the body 2205 may have some slight flexibility, but in practice it is stiff enough to allow the tube 2200 to at least partially insert into the vagina of a woman. In one embodiment the body 2205 may resemble a hollow cylinder having an annular cross-section but this is for purpose of illustration only. For example, in another embodiment the body 2205 may resemble a hollow truncated cone wherein the sides of the body 2205 are not parallel. In yet another embodiment a cross-section of the body 2205 may have another geometric shape such as, but not limited to, square, hexagonal, or octagonal.

In example embodiments, the body 2205 may have a first end 2210 which attaches to the bulb 2100. For example, in one nonlimiting example embodiment, the first end 2210 of the body 2205 may include protrusions 2212 which may complement recesses 2112 formed at a first end 2110 of the bulb 2100 as shown in at least FIG. 33. In some example embodiments an adhesive may be provided between the protrusions 2212 and the recesses 2112 to enable a secure connection between the bulb 2100 and the tube 2200. In one nonlimiting example embodiment, the protrusions 2212 may resemble, but are not required to resemble, rings extending from the body 2205 of the tube 2200 and the recesses 2112 may be formed to receive the ringlike protrusions 2212. In other embodiments the protrusions 2212 are omitted entirely and the first end 2210 of the body 2205 may simply insert into the first end 2110 of the bulb 2100. In yet another embodiment, the first end 2110 of the bulb 2100 may include protrusions rather than recesses and the first end 2210 of the body 2205 may include recesses rather than protrusions. In this latter embodiment, the recesses of body 2205 are configured to compliment and receive the protrusions of the bulb 2100.

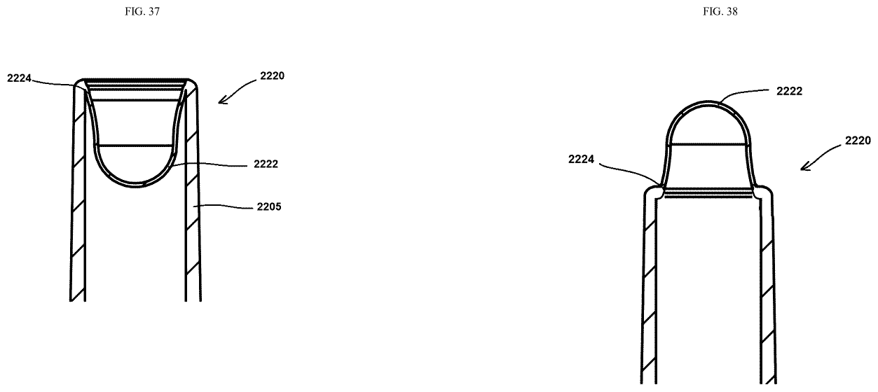

In example embodiments, the tube 2200 may have a flexible member 2222, for example, a flexible membrane, arranged at a second end 2220 of the body 2205. The flexible member 2222 may be configured to receive medication, whether in cream, liquid, or solid form (for example, in the form of a pill). In one nonlimiting example embodiment, the body 2205 of the tube 2200 and the flexible member 2222 of the tube 2200 are formed from a continuous material where a thickness of the body 2205 reduces at the second end 2220 of the body 2205 to form a hinge 2224 for the flexible member 2222. In FIG. 37 the flexible member 2222 is shown residing within the body 2205 of the tube 2200 whereas FIG. 38 illustrates the flexible member 2222 flexing outwards and out of the body 2205 of the tube 2200. While the particular example embodiment described above illustrates the body 2205 and the flexible member 2222 being formed from a continuous material, the invention is not limited thereto. For example, in another nonlimiting example embodiment the flexible member 2222 may be separately formed and attached to the second end 2220 of the body 2205 by a conventional means such as, but not limited to, an adhesive.

In example embodiments the applicator 2000 may form a substantially, if not completely, airtight structure. In a first state, when the bulb 2100 is in an uncompressed and/or undeformed state, the flexible member 2222 of the tube 2200 is inverted as shown in at least FIG. 29. In this state, the flexible member 2222 forms a pocket into which medication may be placed. Thus, the flexible member 2222 may function as a medication chamber. However, when the bulb 2100 is pressed pressure exerted against the flexible member 2222 causes the flexible member 2222 to invert, as shown in at least FIG. 30, to allow expose and/or dispense any medication that may be in the flexible member 2222. When the bulb 2100 returns to an uncompressed state (for example, when a user ceases to press the bulb 2100), the bulb 2100 expands back to its uncompressed state causing the flexible member 2222 to once again invert to its first state as shown in at least FIG. 29.

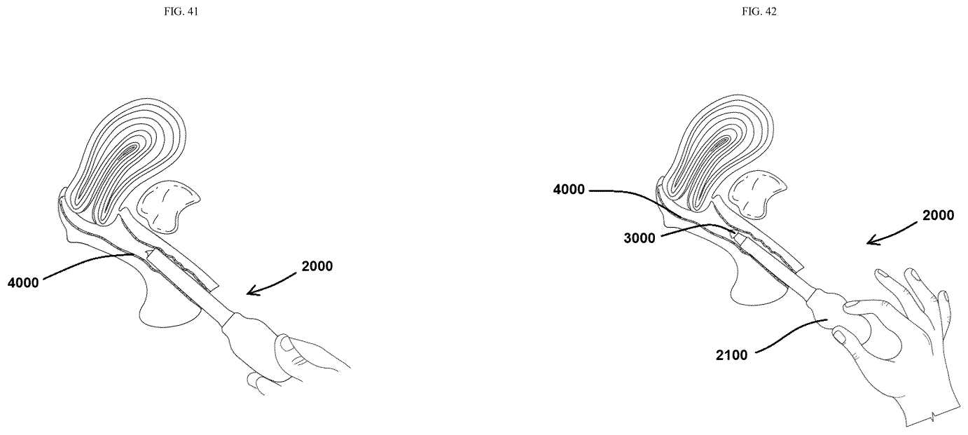

FIGS. 39-42 illustrate a use case for the intravaginal medication applicator 2000. As shown in FIGS. 39 and 40, a user may apply medication 3000 to the flexible member 2222 of the applicator 2000 when the applicator 2000 is in the first state. After the medication 3000 is provided to the flexible member 2222, tube 2200 of the applicator 2000 may be inserted into a vagina 4000 as shown in FIG. 41. Once in a proper location, the bulb 2100 may be pressed causing the flexible member 2222 of the applicator 2000 to invert and apply medication inside the vagina 4000, as shown in FIG. 42. In some embodiments a woman may twist or provide side to side motion to the applicator 2000 so that it will act like a human finger and “rub” the cream more evenly into the vaginal tissue reducing the chance that the cream will release out of the vagina in a large quantity. This aspect of this nonlimiting example embodiment reduces and/or eliminates the problem associated with conventional applicators which, when estrogen is applied, may form balls, chunks, lumps, or clumps of cream that can sometimes release out of the vagina in one piece and is messy. Although the above operations illustrate an exemplary method of applying medication to a vagina, this is not meant to limit the invention as the inventive concepts may be implemented in other ways including, but not limited to, applying medication inside a person's rectum, throat, nose and/or ears. Furthermore, while the exemplary applicator 2000 is shown as being comprised of a separate tube 2200 and bulb 2100 an additive or casting process may be used such that the tube 2200 and the bulb 2100 actually form an integral structure.

Example embodiments of the invention have been described in an illustrative manner. It is to be understood that the terminology that has been used is intended to be in the nature of words of description rather than of limitation. Many modifications and variations of example embodiments are possible in light of the above teachings. Therefore, within the scope of the appended claims, the present invention may be practiced otherwise than as specifically described.

1. An applicator comprising:
a tube having a stiff body and a flexible membrane, where the body includes a first end and a second end, where the first end of the body is open and the flexible membrane is arranged at the second end of the body, and where the flexible membrane may be in a first state where the flexible membrane resides in the body and a second state where the flexible membrane resides outside of the body; and
a bulb attached to the second end of the body, wherein when the bulb is squeezed air in the applicator is moved against the flexible membrane to cause the flexible membrane to be in the second state and when the bulb is released air moves back into the bulb causing the flexible membrane to invert back to the first state.

2. The applicator of claim 1, wherein the tube and bulb form an airtight structure.

3. The applicator of claim 1, wherein the tube and the bulb are in fluid communication with one another.

4. The applicator of claim 1, wherein the body and the membrane are continuous.

5. The applicator of claim 4, wherein the second end of the body is configured as a hinge to allow the flexible membrane to move from the first state to the second state.

6. The applicator of claim 1, wherein the flexible membrane is configured to receive a medication when the flexible membrane is in the first state.

7. The applicator of claim 1, wherein the medication includes at least one a hormone, an analgesic, estrogen, testosterone, progesterone, and dehydroepiandrosterone (DHEA).

8. The applicator of claim 1, wherein the flexible membrane is configured to receive at least one of a lubrication, moisturizer, antifungal, antibacterial, and local anesthetic when the flexible membrane is in the first state.

9. The applicator of claim 1, wherein the flexible membrane is configured to receive at least one of a cream, liquid, tablet, capsule, and suppository.

10. The applicator of claim 1, wherein the flexible membrane is attached to the second end of the body.

11. A method of applying medication comprising:
obtaining an applicator having a tube and a bulb, wherein the tube has a body having a first end connected to the bulb and a second end with a flexible membrane and wherein the flexible membrane may be in a first state where the flexible membrane resides in the body and a second state where the flexible membrane resides outside of the body; and
placing medication on the flexible membrane while the flexible membrane resides in the body;
inserting the applicator into a vagina; and
inverting the flexible membrane to deposit the medication inside the vagina.

12. The method of claim 11, further comprising:
squeezing the bulb, wherein the flexible membrane is inverted in response to the bulb being squeezed.

13. The method of claim 11, wherein when the applicator is inserted into the vagina the bulb remains exposed to allow a user to squeeze the bulb to deposit the medication inside the vagina.

14. The method of claim 11, wherein the applicator is airtight.

15. The method of claim 11, wherein the medication includes at least one a hormone, an analgesic, estrogen, testosterone, progesterone, and dehydroepiandrosterone (DHEA).
